(19) United States
(12) Patent Application Publication
(10) Pub. No.: US 2016/0030418 A1
(43) Pub. Date: Feb. 4, 2016

(54) METHOD FOR DECREASING LOW DENSITY LIPID PROTEIN

(71) Applicant: Ru-Band Lu, Tainan (TW)

(72) Inventor: Ru-Band Lu, Tainan (TW)

(21) Appl. No.: 14/807,927

(22) Filed: Jul. 24, 2015

Related U.S. Application Data
(60) Provisional application No. 62/031,825, filed on Jul. 31, 2014.

(51) Int. Cl. A61K 31/485

(52) U.S. Cl. CPC A61K 31/485

(57) ABSTRACT

The present invention relates to a method of decreasing cholesterol or low density lipid protein in a subject in need thereof comprising: administering to said subject a pharmaceuticaly effective amount of a composition comprising dextromethorphan.

The present invention also relates to a method of treating a subject suffered from bipolar II disorder, comprising administering to said subject a therapeutically effective amount of valproic acid and at least one agent selected from the group consisting of dextromethorphan and memantine, wherein the dose of dextromethorphan or memantine is a low dose not sufficient to work as a N-methyl-D-aspartate (NMDA) receptor antagonist.

The present invention further relates to a composition for treating bipolar II disorder, which comprises valproic acid and at least one agent selected from the group of dextromethorphan and memantine, wherein the dose of dextromethorphan or memantine is a low dose not sufficient to work as a N-methyl-D-aspartate (NMDA) receptor antagonist.

### CROSS-REFERENCES TO RELATED APPLICATIONS

The present application claims priority to U.S. Provisional Application No. 62/031,825 filed on Jul. 31, 2014, incorporated herein by reference in its entirety.

### FIELD OF THE INVENTION

The present invention relates to novel uses of dextromethorphan and memantine.

### BACKGROUND OF THE INVENTION

Bipolar disorder (BP) characterized by a dysregulation of mood, impulsivity, risky behavior, and interpersonal problems, is a recurrent and often chronic psychiatric illness. According to the World Health Organization (WHO), BP is the sixth leading cause of disability-adjusted life years worldwide among persons aged 15 to 44 years. It is associated with functional impairment, elevated suicide rates, and utilization of mental health systems.

BP is known for recurrent depressive, manic, and mixed episodes, and BP is divided into several categories. However, BP is commonly under-recognized, even in psychiatric settings. Increasing evidences suggest that neuronal degeneration may relate to the etiology and progression of bipolar disorder. Imaging studies suggest that ongoing neuronal atrophy accompanies the disorder. For instance, PET images of the cerebral blood flow and the rate of glucose metabolism, both indicative of brain activity, detect a reduced activity in the subgenual prefrontal cortex during the bipolar depression. This decrement in activity in part corresponds to a reduction of cortical volume, similar in a manner to that seen in magnetic resonance imaging demonstrating the reduced mean volume on grey matters. In BP, abnormalities of the third ventricle, frontal lobe, cerebellum, and possibly the temporal lobe are also noted. These observations suggest a neuronal dysfunction and possibly neuronal loss may be involved in the pathogenesis of BP. Therefore, BP may represent a neuro-degeneration disease.

In addition, BP may be associated with induction of a lot of endotoxins and exotoxins which may increase neurotoxins as well as decrease of neurotrophic factors duo to overactivate microglial cell and inhibit astroglia cell. Those effects may induce pre-inflammatory factors such as TNF-α, C-reactive protein, interleukins, etc. and decrease BDNF etc. which will cause neuron damage or necrosis. The vicious cycle will lead to progressive worsening of the disease.

While the pharmacological guidelines for treatment are well established, treatment for BP remains less than ideal. Most individuals still have breakthrough episodes or significant residual symptoms while on medication. In addition, functional deficits often remain even when patients are in remission. Moreover, most BP patients are newly diagnosed who have not taken any mood stabilizer or antipsychotics in the past. Because many patients with BP remain symptomatic even when fully adherent to their medication regimens, greater understanding of the pathogenesis of this illness and novel treatment modality other than current regimen of mood stabilizers and antipsychotics is needed.

Cholesterol is an organic molecule. It is a lipid molecule and is biosynthesized by all animal cells because it is an essential structural component of all animal (not plant or bacterial) cell membranes that is required to maintain both membrane structural integrity and fluidity. Cholesterol travels through bloodstream in small packages called lipoproteins. These packages are made of fat (lipid) on the inside and proteins on the outside. Two kinds of lipoproteins carry cholesterol throughout body: low-density lipoproteins (LDL) and high-density lipoproteins (HDL). Having healthy levels of both types of lipoproteins is important. LDL cholesterol sometimes is called “bad” cholesterol. A high LDL level leads to a buildup of cholesterol in arteries. HDL cholesterol sometimes is called “good” cholesterol. This is because it carries cholesterol from other parts of body back to liver, so that liver can remove the cholesterol from body. High cholesterol level increases the risk of some conditions, such as coronary heart disease, stroke, peripheral vascular disease, type 2 diabetes, and high blood pressure. Therefore, a method for decreasing cholesterol or LDL level in body is needed.

Dextromethorphan (DM) has been used as an antitussive drug with little side effect for more than 50 years in clinics. It is known to have a wide margin of safety. DM has been shown to be neuroprotective in rodents and protects the 1-methyl-4-phenyl- 1,2,3,6-tetrahydropyridine (MPTP)- or lipopolysaccharide (LPS)-induced dopaminergic neuronal damage in vitro and in vivo. Furthermore, DM also protects against the ischemia-induced neuronal damage in rats and has been reported to have neuroprotective effect on monoamine neurons and to have protective effect against endotoxicity and extratoxicity. DM has neuronal protective effects in high dose and low dose. However, the high dosage of DM may cause side effects including body rash/itching, nausea, vomiting, drowsiness, dizziness, constipation, sedation, confusion, nervousness, and closed-eye hallucination. The high dosage of DM may not only increase side effect but also confuse the detailed mechanism of medication; the low dosage will demonstrate the specific effect of the DM in the decrease of cytokine including TNF-a and neuroprotective effect. The mechanism of the neuroprotective effects of low dose DM is associated with the inhibition of microglia activation but not with its N-methyl-D-aspartate (NMDA) receptor antagonist property. Moreover, a better correlation is found between the anti-inflammatory potency and the neuroprotection of low dose DM. These results suggest that the neuroprotection provided by low dose DM in the inflammation-related neurodegenerative models is not mediated through the NMDA receptor. U.S. Pat. No. 8785472 (Publication No. 2011/0281904 A1) suggests that DM is effective in the treatment of BP patients. In addition, low plasma levels of DM (5-100 ng/ml) found in these patients suggests that this beneficial effect of low dose DM is not mediated through the blockade of NMDA receptors. The clinical studies have indicated that patients taking 30-60 mg/70 kg of DM showed plasma DM concentrations of about 10-30 ng/ml (28-84 nM) which is not effect in NMDA receptor. The same low dose of DM causes a significant anti-inflammatory as well as a BDNF-increasing effect in bipolar disorder patients.

Memantine used to be recognized as a noncompetitive N-methyl-D-aspartate receptor antagonist. It was found to have neuroprotective effect in several neurodegenerative diseases in the past years. Memantine can inhibit brain inflammatory response through its action on neuroglial cells and provide neurotrophic effect. Mechanistic studies reveal that the high potency of small dosage of memantine is due to its dual actions: an anti-inflammatory effect by reducing the activity of microglia and an increase in the release of neurotrophic factors, such as BDNF, GDNF by astroglia (U.S. patent application Ser. No. 12/486,630 (Publication No. 2009/0253803 A1). It is also found that even 1/100 dosage of memantine (0.2 mg/kg) may be effective in opioid addictive behavior in rat by conditioned place preference.

### BRIEF DESCRIPTION OF THE DRAWINGS

FIG. 1 shows decreasing tendency of total cholesterol over 4-week of treatment (194.70±39.49 vs. 186.16±37.95 mg/dl; P=0.09).

FIG. 2 shows decreasing tendency of low density lipid protein (LDL) over 4-week of treatment (127.70±34.24 vs. 120.84±34.013 mg/dl; P=0.13).

### SUMMARY OF THE INVENTION

The present invention relates to a method of decreasing cholesterol or low density lipid protein in a subject in need thereof comprising: administering to said subject a pharmaceuticaly effective amount of a composition comprising dextromethorphan.

The present invention also relates to a method of treating a subject suffered from bipolar II disorder, comprising administering to said subject a therapeutically effective amount of valproic acid and at least one agent selected from the group consisting of dextromethorphan and memantine.

The present invention further relates to a composition for treating bipolar II disorder, which comprises valproic acid and at least one agent selected from the group of dextromethorphan and memantine.

### DETAILED DESCRIPTION OF THE INVENTION

The present invention is to investigate the efficacy of combination therapy of add-on low dose dextromethorphan (DM) and memantine, add-on DM, add-on memantine and placebo for bipolar II disorder (BP-II) treatment in a randomized, double-blind, placebo-controlled trial and will explore inflammatory and neurodegeneration to identify biological markers for predicting treatment response.

Also, the present invention is to investigate whether adding on dextromethorphan and memantine to valproate treatment has synergistic effect and show higher efficacy in improving symptomatology, anti-inflammatory factors, and plasma BDNF in bipolar disorder or other major mental illnesses patients.

It is predicted that patients with altered expressions of inflammatory and neurodegeneration factors (e.g. BDNF, TNF-α, . . . ), lower plasma levels of BDNF, higher cytokine levels, and more severe baseline symptoms are more likely to benefit from such combination therapy.

The present invention also finds that dextromethorphan can decrease cholesterol or low density lipid protein in human.

Unless otherwise defined in the present context, the scientific and technical terms used in the present invention should possess meaning commonly known by any person with ordinary skill in the art. The meaning and scope of the terms should be clear; nevertheless, in any circumstance of discrepancy in the meaning, definition provided in the present context precedes those defined in any other dictionaries or external references.

The entire context of any references cited in the present context is incorporated into the present context as references.

Unless otherwise needed in the present context, singular terms should include plural forms and plural terms should include singular forms.

As used herein, the term “suppress”, “decrease” or “reduce” or any variation of these terms when used in the claims and/or specification includes any measurable reduction or complete inhibition to achieve the desired result.

When the term “one” is used in conjunction with the term “comprising” in claims and/or specification, it means “one”, but also equivalent to the meaning of “one or more”, “at least one” and “one or more than one”.

The term “or” used in the claims refers to “and/or” unless substitutes are mutually exclusive.

The term “about” in the full text of the present application refers to a value comprising inherent error differences derived from the methods or devices used for determining the value, or differences exist in the subjects.

As used in the specification and claims, the term “comprise” (and any form of comprise), “have” (and any form of have), “include” (and any form of include), or “contain” (and any form of contain) indicates inclusion rather than limitation. When a patent claim states that a particular product or method comprises certain elements, this means that other elements may also be present.

Therefore, the present invention provides a method for decreasing cholesterol or low density lipid protein in a subject in need thereof comprising: administering to said subject a pharmaceutically effective amount of a composition comprising dextromethorphan. This method can be used in treating diseases or conditions associated with high cholesterol, such as, for example, coronary heart disease, stroke, peripheral vascular disease, type 2 diabetes, high blood pressure, or hypercholesterolemia. In an embodiment, the dose of dextromethorphan is a low dose not sufficient to work as an N-methyl-D-aspartate (NMDA) receptor antagonist. For example, the dose of dextromethorphan may equal to or less than 60 mg/day. In an embodiment, the dose of dextromethorphan ranges from 10 mg/day to 60 mg/day. In an embodiment, the dose of dextromethorphan ranges from 30 mg/day to 60 mg/day.

The present invention also provides a method of treating a subject suffered from bipolar II disorder, comprising administering to said subject a therapeutically effective amount of valproic acid and at least one agent selected from the group consisting of dextromethorphan and memantine, wherein the dose of dextromethorphan or memantine is an ultra low dose not sufficient to work as an N-methyl-D-aspartate (NMDA) receptor antagonist. For example, the ultra low dose of memantine for human may range from about 1 mg/day to 10 mg/day, and the ultra low dose of dextromethorphan for human may range from about 10 mg/day to 60 mg/day. In an embodiment, the method administers to said subject a therapeutically effective amount of valproic acid and a combination of dextromethorphan and memantine. The combination of dextromethorphan and memantine produces a synergistic effect in treating bipolar II disorder. The ratio of dextromethorphan to memantine can be from 12:1 to 3:1. In an embodiment, the ratio of dextromethorphan to memantine is 6:1.

The present invention further provides a composition for treating bipolar II disorder, which comprises valproic acid and at least one agent selected from the group of dextromethorphan and memantine, wherein the dose of dextromethorphan or memantine is an ultra low dose not sufficient to work as an N-methyl-D-aspartate (NMDA) receptor antagonist. For example, the ultra low dose of memantine for human may range from about 1 mg/day to 10 mg/day, and the ultra low dose of dextromethorphan for human may range from about 10 mg/day to 60 mg/day. In an embodiment, the composition comprises valproic acid and a combination of dextromethorphan and memantine. The ratio of dextromethorphan to memantine in the composition can be from 12:1 to 3:1. In an embodiment, the ratio of dextromethorphan to memantine is 6:1.

The composition of the present invention further comprises a pharmaceutical carrier, buffer, diluent, or excipient. The suitable diluents are polar solvents, such as water, alcohol, ketones, esters and mixtures of the above solvents, preferably water, alcohol and water/alcohol mixture. For the preferable embodiment, the suitable solvents are water, normal saline, buffering aqueous solution and buffering saline etc. The excipients used with the composition of this invention can be in liquid, semi-liquid or solid form, such as lactose, dextrin, and starch and sodium stearate. Liquid excipients include water, soybean oil, wine and juices etc.

The composition of the present invention can be administered by oral or injection. The composition can be taken by oral in liquid, semi-liquid or solid form. The composition provided by injection is in liquid or semi-liquid form. The injection includes intravenous injection, the abdominal cavity and intramuscular injection.

### EXAMPLES

The examples below are non-limiting and are merely representative of various aspects and features of the present invention.

### Example 1

250-300 male and female BP-II patients (18-65 years old) were recruited, from the Department of Psychiatry at National Cheng Kung University Hospital and Tri-Service General Hospital into a 12-week treatment. The research protocol was examined for approval by the Institutional Review Board for the Protection of Human Subjects at National Cheng Kung University Hospital and Tri-Service General Hospital. All participants were given a complete description of the study and were asked to sign an informed consent before the study began. Patients who had major, mental illnesses other than BP-II and who had taken memantine or DM within 1 week before the first dose of the double-blind medication were excluded. The Chinese Version of Modified Schedule of Affective Disorder and Schizophrenia-Life Time (SADS-L) will be conducted for confirmation of the diagnosis. Although DSM-IV-TR (American Psychiatric Association, 2000) criteria require a minimum duration of 4 days of hypomania, current epidemiologic data suggest that a 2-day duration is more prevalent in community samples; therefore, the 2-day minimum for hypomania in the diagnosis of BP-II was used in this example. After each patient had been diagnosed based on DSM-IV criteria by a senior psychiatrist and confirmed by a clinical psychologist using a structured interview, SADS-L, the severity of each patient's clinical symptoms was assessed using the 17-item Hamilton Rating Scale for Depression (HRSD) and the Young Mania Rating Scale (YMRS). All the patients at Screen who score 18 on the HRSD or 14 on the YMRS were recruited. Eligible patients were randomly assigned into four treatment groups (n=40-45 for each group) in a double-blind manner and all groups matched in demographic characteristics, symptom severity, while they received open-label valproic acid (VPA) treatment (500 mg and 1000 mg daily [50-100 g/ml in plasma]): 1) DM (30 mg/day), 2) memantine (5 mg/day), 3) combination of DM and memantine (30 mg/day DM and 5 mg/day memantine), and 4) placebo respectively for 12 weeks. Patients were randomized into 4 groups through a computer-generated randomization table to receive placebo or any of active treatments in a 1:1:1:1 ratio. The patients with side-effect intolerance or clinical worsening were withdrawn earlier.

### Patient Selection

Inclusion Criteria

1) Male or female patient aged ≧18 and ≦65 years.

2) Signed informed consent by patient or legal representative.

3) The Chinese version of the modified SADS-L, a semi-structured interview aimed at formulating the main bipolar II diagnoses based upon DSM-IV criteria with good inter-rater reliability. This modified version of SADS-L with its diagnoses was used as the gold standard.

4) A 2-day minimum for hypomania to diagnose BP-II.

5) A total of Hamilton Rating Scale for Depression (HDRS) scored at least 18 or Young Mania Rating Scale (YMRS) scored at least 14 at the screening stage.

6) Patient or a reliable caregiver was expected to ensure acceptable compliance and visit attendance for the duration of the study.

Exclusion Criteria

1) Females who are pregnant or nursing.

2) Women of childbearing potential not using adequate contraception as per investigator judgment or not willing to comply with contraception for duration of study.

3) Patient had received DM, memantine, other anti-inflammatory medication within 1 week prior to first dose of double-blind medication, such as cyclo-oxygenase 2 (Cox-2) inhibitors.

4) Clinically significant medical condition e.g., cardiac, hepatic and renal disease with current evidence of poor controlled.

5) Patient had received electroconvulsive therapy (ECT) within 4 weeks prior to the first dose of double-blind medication.

6) Increase in total SGOT, SGPT, BUN and creatinine by more than 3× upper limit of normal.

Measures of Symptomatology

Efficacy will be assessed by the YMRS, HDRS and Side-Effects.

Clinical ratings were performed by research psychiatric professionals who were well-trained and experienced in the rating scales. All assessments were blind to treatment assignment and completed at baseline and at the end of week 1, 2, 4, 8 and 12. Routine laboratory tests, including CBC and biochemistry were checked at baseline and the end of week 12.

Diagnostic and Symptom Evaluation Tools

(1) Chinese Version of the Modified Schedule of Affective Disorder and Schizophrenia-Lifetime (SADS-L)

The Chinese version of the modified SADS-L was a semi-structured interview aimed at formulating the main diagnosis based upon DSM-IV criteria. We used this modified version of the SADS-L as the gold standard for diagnosis.

(2) Young Mania Rating Scale (YMRS) and 17-item Hamilton Depression Rating Scale (HDRS)

Those clinically suspected of having a mood disorder were invited to be interviewed with the Chinese version SADS-L and diagnosed as having BP-II. The severity of current symptoms was assessed by using the YMRS and HDRS. The diagnosis of a mood disorder was made according to DSM-IV criteria, except for BP-II, for which we used a 2-day instead of a 4-day duration for hypomania.

(3) Side-Effects Checklist

The severity of side effects was evaluated by using the Chinese version of Side-Effects Checklist which derived from Psychiatric Rating and Diagnostic Instruments (TRIMS, 1972) and included 7 categories as follows: 1) Mental Status; 2) Genito-Urinary; 3) Cardiovascular; 4) Head-Neck; 5) Extremities; 6) Skin; 7) Gastrointestinal. The order of severity was 3-point: 0=Not present; 1=Mild or occasional; 2=Moderated or occurs several times a day; 3=Severe or persistent.

### Laboratory Assessments

Serum levels of DM, memantine, plasma BDNF, and cytokines were measured. Whole blood was draw and collected from the antecubital vein. Plasma were isolated from the whole blood after the 3000 g centrifugation at 4° C. for 15 min and stored at −80° C. immediately. Plasma BDNF, CRP, TNF-α, and interleukin (IL-8) were measured at baseline, 1, 2, 4, 8 and 12 weeks and quantified using the enzyme-linked immunosorbent assays (ELISAs). The Quantikine Human cytokine kit (R&D system), and Molecular Devices (SpectraMax-M2) ELISA reader were used to analyze the plasma cytokine levels. The low limit sensitivity was 0.5 pg/ml, 0.125 pg/ml, 0.5 pg/ml and 62.5 pg/ml for TNF-α, IL-1β, IL-8 and BDNF respectively. Finally, HPLC with UV detector and therapeutic drug monitoring (TDM) was used to measure the plasma DM and VPA level respectively.

### Statistical Analysis

The analyses were conducted using SPSS.18.0 windows version.

1. Participants who completed this study for at least 8 weeks of treatment, the data was conducted using Intent to Treatment (ITT) and last observation carried forward (LOCF).

2. Demographic characteristics were analyzed using Chi-Square for the nominal variables.

3. Multi-variate analyses: applying multiple regression and logistic regression to analyze each variable (including demographics, symptom severity, plasma BDNF, cytokines, change of lipid profiles and response to medication treatment) in prediction of prognosis in patients with BP-II.

4. The effect size conventions in power analysis were determined according to Buchner et al. (1996) as follows: small effect size=0.10, medium effect size=0.30, large effect size=0.50 for the test; and small effect size=0.02, medium effect size=0.15, large effect size=0.35 for the multiple regression model (alpha=0.05).

### Example 2

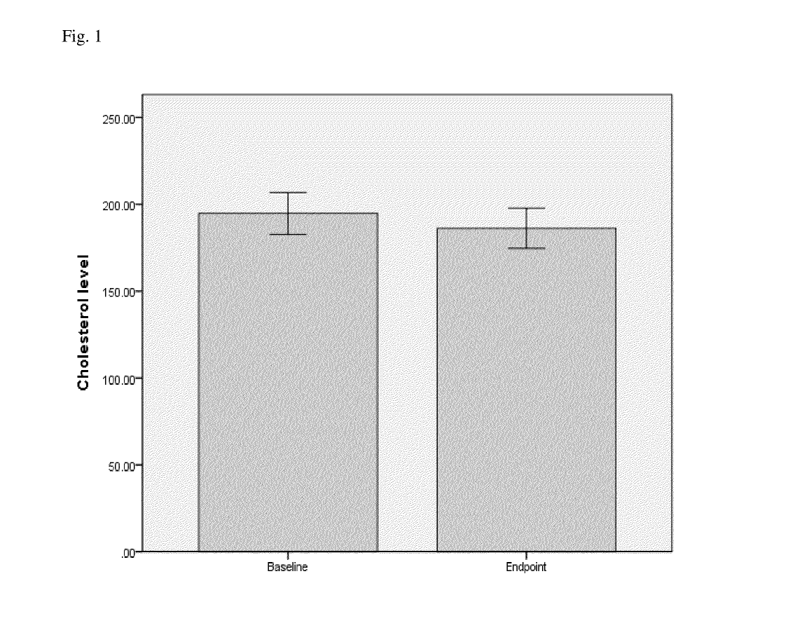

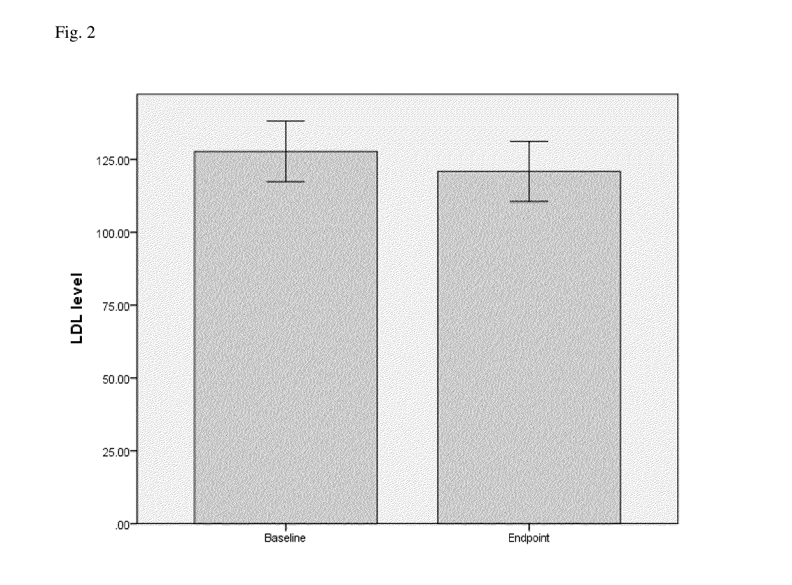

This example was a clinical trial for the treatment of bipolar II disorder using 30 mg/day of dextromethorphan (off label use) adding on to depakine (plasma level 50-100 ug/dl). 44 patients with bipolar II disorder were recruited, and change of cholesterol was monitored only over 4 weeks. The decreasing tendency of total cholesterol over 4-week of treatment (194.70±39.49 vs. 186.16±37.95 mg/dl; P=0.09) (FIG. 1), as well as the low density lipid protein (LDL) (127.70±34.24 vs. 120.84±34.013 mg/dl; P=0.13) (FIG. 2) was found. It showed that dextromethorphan decreased cholesterol and LDL in human, thus had great potential in treating coronary heart disease, stroke, peripheral vascular disease, type 2 diabetes, high blood pressure, or hypercholesterelmia.

One skilled in the art readily appreciates that the present invention is well adapted to carry out the objects and obtain the ends and advantages mentioned, as well as those inherent therein. The compositions, processes and methods for producing them, and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for decreasing low density lipid protein in a subject in need thereof comprising: administering to said subject a pharmaceuticaly effective amount of a composition comprising dextromethorphan.

2. The method of claim 1, wherein the dose of dextromethorphan is a low dose not sufficient to work as an N-methyl-D-aspartate (NMDA) receptor antagonist.

3. The method of claim 1, wherein the dose of dextromethorphan is equal to or less than 60 mg/day.

4. The method of claim 1, wherein the dose of dextromethorphan ranges from 10 mg/day to 60 mg/day.

5. The method of claim 1, wherein the dose of dextromethorphan ranges from 30 mg/day to 60 mg/day.

6. The method of claim 1, which is further used for decreasing cholesterol in a subject in need thereof.

7. The method of claim 1, which is used in treating diseases or conditions associated with high cholesterol.

8. The method of claim 7, wherein the diseases or conditions associated with high cholesterol comprise coronary heart disease, stroke, peripheral vascular disease, type 2 diabetes, high blood pressure, and hypercholesterelmia.
